(19) United States
(12) Patent Application Publication
(10) Pub. No.: US 2026/0240455 A1
(43) Pub. Date: Aug. 20, 2026

(54) SYSTEMS AND METHODS FOR HUMAN GAIT ANALYSIS, REAL-TIME FEEDBACK AND REHABILITATION USING AN EXTENDED-REALITY DEVICE

(71) Applicant: Magic Leap, Inc., Plantation, FL (US)

(72) Inventors: Emilio Patrick SHIRONOSHITA, Coral Gables, FL (US); Colby Nelson LEIDER, Coral Springs, FL (US); Jennifer Miglionico ESPOSITO, Rapid City, SD (US); Edward NYMAN, JR., Maumee, OH (US)

(73) Assignee: Magic Leap, Inc., Plantation, FL (US)

(21) Appl. No.: 18/874,493

(22) PCT Filed: Jun. 13, 2022
(86) PCT No.: PCT/US2022/072909

§ 371 Date: Dec. 12, 2024

(51) Int. Cl. A61B 5/11; A61B 5/00; G06V 40/20; G16H 50/20

(52) U.S. Cl. CPC A61B 5/112; A61B 5/1114; A61B 5/6803; G06V 40/25; G16H 50/20

(57) ABSTRACT

System and methods for performing gait analysis diagnosis, and rehabilitation and real-time feedback for treating gait disorders using extended reality, such as augmented reality. Image data is captured using native camera(s) on an extended reality headset worn by a subject while walking. The images are analyzed using a location and mapping algorithm to determine head-pose data regarding a position and location of the head of the subject. One or more gait attributes of a gait of the subject by analyzing the head-pose data using a gait-metric prediction algorithm. The gait attributes are analyzed to determine gait disorders, rehabilitation treatment, rehabilitation assessments, and/or rehabilitation feedback.

### FIELD OF THE INVENTION

The present disclosure relates to extended reality systems, and more particularly, to systems and methods for performing gait analysis, diagnosis, and rehabilitation and real-time feedback for treating gait disorders using extended reality technology.

### BACKGROUND

Modern computing and display technologies have facilitated the development of systems for so called “virtual reality” (VR), “augmented reality” (AR), and/or “mixed-reality” (MR) experiences, wherein digitally reproduced images, or portions thereof, are presented to a user in a manner wherein they seem to be, or can be perceived as, real. A VR scenario typically involves presentation of digital or virtual image information without transparency to actual real-world visual input. An AR scenario typically involves presentation of digital or virtual image information as an augmentation to visualization of the real-world around the user (i.e., transparency to real-world visual input). Accordingly, AR scenarios involve presentation of digital or virtual image information with transparency to the real-world around the user. An MR scenario is a version of an AR scenario, except with more extensive merging of the real world and virtual world in which physical objects in the real world and virtual objects may co-exist and interact in real-time. As used herein, the terms “extended reality” and “XR” are used to refer collectively to any of VR, AR and/or MR. In addition, the term “AR” means either, or both, AR and MR.

Various optical systems generate images at various depths for displaying XR scenarios. Some such optical systems are described in U.S. Utility patent application Ser. No. 14/555,585 filed on Nov. 27, 2014 (attorney docket number ML.20011.00), the contents of which are hereby expressly and fully incorporated by reference in their entirety, as though set forth in full.

XR systems typically employ wearable display devices (e.g., head-worn displays, helmet-mounted displays, or smart glasses) that are at least loosely coupled to a user's head, and thus move when the user's head moves. If the user's head motions are detected by the display device, the data being displayed can be updated to take the change in head pose (i.e., the orientation and/or location of user's head) into account.

As an example, if a user wearing a head-worn display device views a virtual representation of a virtual object on the display device and walks around an area where the virtual object appears, the virtual object can be rendered for each viewpoint (corresponding to a position and/or orientation of the head-worn display device), giving the user the perception that they are walking around an object that occupies real space. If the head-worn display device is used to present multiple virtual objects at different depths, measurements of head pose can be used to render the scene to match the user's dynamically changing head pose and provide an increased sense of immersion. However, there is an inevitable lag between rendering a scene and displaying/projecting the rendered scene owing to the complexity of computation involved.

Head-worn display devices that enable AR provide concurrent viewing of both real and virtual objects. With an “optical see-through” display, a user can see through transparent (or semi-transparent) elements in a display system to directly view the light from real objects in a real-world environment. The transparent element, often referred to as a “combiner,” superimposes light from the display over the user's view of the real world, where light from the display projects an image of virtual content over the see-through view of the real objects in the environment. A camera may be mounted onto the head-worn display device to capture images or videos of the scene viewed by the user.

A person's gait (i.e., a person's manner and/or pattern of walking) involves balance and coordination of muscles. There are a number of reasons that may cause a person to have an abnormal gait. Common causes include traumatic injury, a major surgery involving the lower extremities, an inner ear disorder, congenital abnormalities in the lower extremities, neurological disorders, diseases causing neurological disorders (e.g., a stroke or Parkinson's disease), among others. To diagnose and treat (e.g., rehabilitate) gait abnormalities, a gait analysis of a patient can be performed. Currently, gait analysis can be performed by manual inspection by a trained clinician, semi-automatically using dedicated computational equipment and sensors applied to the patient, computer vision-based methods, or a combination. Such gait evaluations typically require a visit by the patient to a remotely-located specialized laboratory. Such laboratories are expensive to build and maintain. Based on the gait analyses, healthcare providers (e.g., a clinician, doctor, technician or physical therapist) can follow best practices and prescribe clinical interventions/treatments designed to allow the subject to maximally recover a normal, or at least improved, gait in the shortest time possible. The clinical interventions/treatments typically involve regular meetings with one or more physical therapists who assist in the patient's treatment and recovery.

Although the current practices for gait analysis have proven somewhat useful in evaluating, diagnosing, and prescribing treatment for gait disorders, there is a need for more cost effective, convenient, and/or accurate systems and methods to perform gait analysis and gait-rehabilitation tasks.

### SUMMARY

The present disclosure is directed to systems and methods for performing gait analysis, diagnosis, and rehabilitation and real-time feedback for treating gait disorders using XR (typically AR, as it allows the subject to see the surrounding environment while walking). The systems and methods disclosed herein utilize an XR headset, which can eliminate, or at least reduce, the need for dedicated computational equipment and sensors applied to the subject. The use of more cost-effective, and increasingly available wearable XR devices can both lower the cost of gait analysis, diagnosis and treatment, and facilitate in-home care by allowing clinicians and patients to communicate remotely using the XR devices and telecommunication technology.

In general, the systems and methods are implemented on a computerized extended reality system (XR system). While the systems and methods can be implemented on any type of XR system (including VR, AR, or MR), the systems and methods herein are described with respect to an augmented-reality system (AR system), as it allows the subject to see the surrounding environment through the transparent or semi-transparent headset without having to capture and generate images of the surrounding environment on the display as with a VR system. The AR system comprises a computer having a computer processor, memory, a storage device, and software stored on the storage device and an executable to program the computer to perform operations enabling the AR system. The AR system also includes a wearable AR headset configured to be worn on the head of a subject (e.g., a patient or other user). The AR headset has a display and projection optics configured to project AR images into the eyes of the subject. The AR headset is operably coupled to, and in communication with, the computer. The AR headset has one or more outward facing image sensors (e.g., cameras, or other computer vision devices) for capturing images of the surrounding environment of the user. The AR headset may also include one or more other sensors, such as inward facing cameras (e.g., for eye-tracking, etc.), other camera(s), a computer-vision device, kinematic sensors such as one or more inertial measurement units (IMUs), an accelerometer, a direction sensor, a compass, and/or a gyroscope, and potentially a plurality of other sensors. Typically, the AR system is configured to present 3D virtual images in an AR field of view to the user that simulate accurate locations of virtual objects in a world-coordinate system.

Hence, one embodiment disclosed herein is directed to a computer-implemented method for performing a gait analysis using the AR headset worn on the head of a subject. The method comprises capturing image data from the one or more image sensors disposed on the AR headset as the subject walks. A simultaneous location and mapping (SLAM) analysis is performed on the image data to determine head-pose data regarding a position (i.e., orientation) and location of the head of the subject. For instance, as the subject walks, the subject's head translates in a three-dimensional (i.e., x, y, and z) coordinate space, and also rotates (e.g., tilts) left/right and fore/aft. SLAM techniques are known algorithms well understood by those skilled in the state of the art for constructing and/or updating a map of an environment, including objects in the environment, using image data from image sensors. Thus, in one aspect, the head-pose data may include one or more data representative of the time-dependent, three-dimensional coordinate location of the head, and/or the fore/aft tilt position and/or roll/pitch/yaw of the head and left/right rotational position of the head.

Then, the method uses a gait metric-prediction algorithm to analyze the head-pose data and determine one or more gait attributes (also referred to as gait metrics) of the subject. The gait attributes characterize the subject's gait, such as the step length, step width, step velocity, step quantity, step cadence (steps/unit of time), stance time, gait velocity, gait symmetry, foot pressure, other musculoskeletal kinematic features, etc. This is one highly innovative aspect, in that the presently disclosed systems and methods can determine gait attributes using head-pose data generated from images captured from cameras on readily available AR headsets.

In another aspect of the method, the gait-metric prediction algorithm is a trained model using a deep-learning approach. For instance, in other aspects of the method, the deep-learning approach may utilize a convolutional neural network (CNN) or a long term short term memory (LSTM) neural network. The trained model is trained with training data developed from obtaining time-dependent head-pose data and gait data from a plurality of subjects. Each subject is fitted with head sensors (e.g., cameras on an AR headset) for obtaining head-pose data, and kinematic sensors worn on different parts of the subject's body (e.g., motion sensors on the feet, legs, hips, etc.) for obtaining motion data of the different parts of the subject's body. Each subject then walks and head-sensor data and kinematic sensor data is captured from the head sensors and kinematic sensors. A SLAM analysis is performed on the head sensor data to determine time-dependent head-pose data, as described above. The kinematic sensor data from the kinematic sensors is also analyzed to determine time-dependent gait data (i.e., motion data for the different parts of the subject's body). The head-pose data and gait data, and/or data extracted from the head-pose data and gait data, are then used as training data to train the gait-metric prediction algorithm. For instance, the gait data can be further processed and/or analyzed to extract gait attributes, that are used as part of the training data.

In yet another aspect, the method may further include analyzing the one or more gait attributes to determine a diagnosis of a gait disorder of the subject. For instance, the method may determine that the subject has a limp, or an asymmetrical gait caused by a stroke, injury, orthopedic surgery, etc. This may also include analyzing the one or more gait attributes to determine a course of rehabilitation treatment for treating the gait disorder. As some examples, the treatment may be a course of exercise, physical therapy, or the like, including the exercises, frequency (e.g., once a day, twice a week, etc.), duration of each session (30 minutes, 1 hour, etc.), and length of treatment (e.g., 3 months, 6 months, 1 year, until predetermined level of improvement in certain gait attribute(s), etc.).

In another aspect, the method may also include determining one or more gait classifiers based on the one or more gait attributes. The gait classifiers may include classification such as a gait disorder caused by a neurodegenerative disease, a neurological trauma, a musculoskeletal injury, an orthopedic surgery, or the like.

In still another feature, the method may include cloud-based computing aspects, including transmitting an output based on the one or more gait attributes to a cloud-based computing system for determination of one or more gait classifiers based on the one or more gait attributes; and/or transmitting an output based on the one or more gait attributes to a cloud computing system for determination of a rehabilitation treatment based on the one or more gait attributes. The output may be the one or more gait attributes, or the like. In still another aspect, any of the data generated by the method, including the gait classifiers, gait attributes, gait disorder, etc. may be output to a healthcare provider via electronic communication, such as by a portal, email, or other means.

In still another aspect, the method may also include obtaining data from other sensors in addition to the image sensors. In such cases, the step of determining the one or more gait attributes may also analyze the other sensor data using the gait-metric prediction algorithm. As an example, the method can include obtaining second sensor data from a second sensor. The gait-metric prediction algorithm can analyze both the head-pose data and the second sensor data to determine gait attributes. In additional aspects, the second sensor may be a kinematic sensor or an image sensor, such as one or more of an IMU, an accelerometer, a direction sensor, a compass, a gyroscope, a GPS sensor, a camera, and/or a computer-vision sensor, and may be disposed on the AR headset (e.g., a standard sensor of an available AR headset), or an independent sensor worn on a different part of the subject's body separately from the AR headset, such as on the waist or legs. In another aspect, the head-pose data and the second sensor data may be combined prior to analyzing the head-pose data and second sensor data using the gait-metric prediction algorithm. As an example, the head-pose data and the second sensor data may be combined using a sensor-fusion algorithm that applies different weights to head-pose data and the second sensor data.

In still another aspect, the method may utilize an external image sensor separate from the AR headset to determine body position data that can also be used to determine the gait attributes. The method includes capturing second image data from the external image sensor separate from the AR headset, and performing a SLAM analysis on the image data to determine body position data of the body of the subject. Then, the step of determining the one or more gait attributes includes analyzing the head-pose data and the body position data using the gait-metric prediction algorithm. In another way, the head-pose data and the body position data may be combined prior to analyzing the head-pose data and body position data using the gait-metric prediction algorithm, such as by using a fusion formula which applies different weights to the head-pose data and the body position data.

In yet another aspect, the method may include providing instructions to the subject regarding rehabilitation tasks for a course of rehabilitation treatment for treating the gait disorder. For instance, the method may provide rehabilitation task virtual content regarding the rehabilitation task to the subject via the AR headset. In one aspect, the rehabilitation task virtual content may include video instructions for performing the rehabilitation task presented on the display of the AR headset. In another aspect, the rehab task presentation may include a game-engine based synthetic visual representation of a physical therapist that interacts with the subject via synthetic speech, visual gestures, audio and haptic feedback. In still another option, the rehab task virtual content may assume the form of a live-streamed virtual video of a clinician providing instructions for performing the rehabilitation task presented on the AR headset.

In still another aspect, the method may also include analyzing a subject performing gait rehabilitation (rehab) tasks, assessing the subject's progress and/or providing feedback to the subject. Thus, the method may include capturing rehab image data from the one or more image sensors on the AR headset of the subject performing a gait rehabilitation task for treating a gait disorder (e.g., the gait rehab task determined by the method based on the gait attributes), and performing a SLAM analysis on the rehab image data to determine rehab head-pose data of the head of the subject, same or similar to the SLAM analysis on the original image data. Then, the rehab head-pose data is analyzed using the gait-metric prediction algorithm to determine one or more rehab gait attributes/metrics of a gait of the subject during the rehab tasks. The method then includes analyzing the rehab gait attributes to assess the subject's progress and/or to determine feedback to the subject regarding the subject performing the gait rehabilitation task. The feedback can then be provided to the subject via the AR headset, such as in the form of augmented reality video, audio, and/or haptic feedback, or any of a plurality of other feedback mechanisms.

Another embodiment disclosed herein is directed to an AR system for performing gait analysis, diagnosis, and rehabilitation and real-time feedback for treating gait disorders using AR. The AR system may be the same or similar system which performs the method embodiments described herein. Hence, in one embodiment, the AR system comprises a computer having a computer processor, memory, a storage device, and a software application(s) stored on the storage device and executable to program the computer to perform operations enabling the augmented reality system. The AR system includes an AR headset including a display for displaying 3D virtual images (i.e., AR images). The AR headset includes a frame structure configured to be worn on the head of the subject. For example, the display may include a pair of light projectors, panel displays, or the like, and optic elements to project the 3D virtual images in the AR field of view into the eyes of the user. The headset may also allow a degree of transparency to the real-world surrounding the user such that the AR images augment the visualization of the real-world. The AR headset is operably coupled to, and in communication with, the computer. The AR headset includes one or more outward facing image sensors (e.g., cameras, or other computer vision devices) for capturing images of the surrounding environment of the user. The AR headset may also include one or more other sensors, such as inward facing cameras (e.g., for eye-tracking, etc.), and one or more kinematic sensors (e.g., an inertial measurement unit (IMU), an accelerometer, a direction sensor, a compass, a gyroscope, a GPS sensor, a camera, and/or a computer vision, etc.)

The software application(s) include a gait-analysis software application executable by the computer processor to program the AR system to execute a process for performing gait analysis, diagnosis, rehabilitation and/or real-time feedback for treating gait disorders. For instance, the process may include but it's not limited to the followin: capturing image data from the one or more image sensors headset as the subject walks; performing a SLAM analysis on the image data to determine head-pose data of the head of the subject; and determining one or more gait attributes of a gait of the subject by analyzing the head-pose data using a gait-metric prediction algorithm.

In additional aspects, the AR system may be configured such that the process includes any combination of one or more of the aspects of the method embodiments described herein. For instance, the AR system may be configured to perform the methods including, one or more of: determining a gait disorder; determining a course of rehabilitation treatment, including rehabilitation tasks; providing rehab task virtual content; capturing rehab image data and using the rehab image data for the subject performing a gait rehab task to determine rehab gait attributes; determining feedback regarding the gait rehab task and providing the feedback to the subject; obtaining training data for training the gait-metric prediction algorithm; training the gait-metric prediction algorithm, etc. The AR system includes the components, elements and their arrangement as described for the method embodiments. For instance, the AR system may further include one or more kinematic sensors, external image sensor(s) separate from the AR headset, etc. The AR system may also have communication adapters to communicate via one or more communication networks, including the Internet or a private cloud, to enable the cloud-based functionality of certain aspects of the method embodiments.

Another disclosed embodiment is directed to a non-transitory computer-readable medium having stored thereon a sequence of instructions that, when stored in memory and executed by a processor programs the processor to cause an AR system to perform a process for performing gait analysis, diagnosis, rehabilitation and/or real-time feedback for treating a gait disorder. Accordingly, in one embodiment, the process includes capturing image data from one or more image sensors disposed on the AR headset as the subject walks; performing a SLAM analysis on the image data to determine head-pose data regarding a position and location of the head of the subject; and determining one or more gait attributes of a gait of the subject by analyzing the head-pose data using a gait-metric prediction algorithm.

In additional aspects, the computer-readable medium includes instructions wherein the process includes any combination of one or more of the additional aspects and features of the method embodiments described herein. For instance, the process may including performing one or more of: determining a gait disorder; determining a course of rehabilitation treatment, including rehabilitation tasks; providing rehab task virtual content; capturing rehab image data and using the rehab image data for the subject performing a gait rehab task to determine rehab gait attributes; determining feedback regarding the gait rehab task and providing the feedback to the subject; obtaining training data for training the gait-metric prediction algorithm; training the gait-metric prediction algorithm, among other tasks.

Additional and other objects, features, and advantages of the disclosure are described in the detail description, figures and claims.

### BRIEF DESCRIPTION OF THE DRAWINGS

The drawings illustrate the design and utility of various embodiments of the present disclosure. Note that the figures are not drawn to scale, and that elements of similar structures or functions are represented by like reference numerals throughout the figures. To better appreciate how to obtain the above-recited and other advantages and objects of various embodiments of the disclosure, a more detailed description of the present disclosures briefly described above will be rendered by reference to specific embodiments thereof, which are illustrated in the accompanying drawings. Understanding that these drawings depict only typical embodiments of the disclosure and are not therefore to be considered limiting of its scope, the disclosure will be described and explained with additional specificity and detail through the use of the accompanying drawings in which:

FIG. 1 depicts a user's view of an AR field of view on a 3D display system of an AR system, according to some embodiments.

FIGS. 2A-2B schematically depict an AR system and subsystems thereof, according to some embodiments.

FIG. 3 is a flow chart of a method for developing a gait-metric prediction algorithm using a deep learning model, according to one embodiment.

FIG. 4 is a flow chart of a method for performing a gait analysis using the AR system of FIGS. 2A-2B, according to one embodiment.

FIG. 5 is a flow chart of another method for performing a gait analysis using the AR system of FIGS. 2A-2B, according to an additional embodiment.

FIG. 6 is a flow chart of a method of using the AR system of FIGS. 2A-2B to analyze a subject performing gait rehabilitation tasks, assess the subject's progress, and/or provide feedback to the subject.

### DETAILED DESCRIPTION

The following describes various embodiments of systems and methods for human gait analysis, gait disorder diagnosis, rehabilitation treatment and real-time feedback using extended reality.

Various embodiments will now be described in detail with reference to the drawings, which are provided as illustrative examples of the disclosure to enable those skilled in the art to practice the disclosure. Notably, the figures and the examples below do not limit the scope of the present disclosure. Where certain elements of the present disclosure may be partially or fully implemented using known components (or methods or processes), only those portions of such known components (or methods or processes) that are necessary for an understanding of the present disclosure will be described, and the detailed descriptions of other portions of such known components (or methods or processes) will be omitted so as not to obscure the disclosure. Further, various embodiments encompass present and future known equivalents to the components referred to herein by way of illustration.

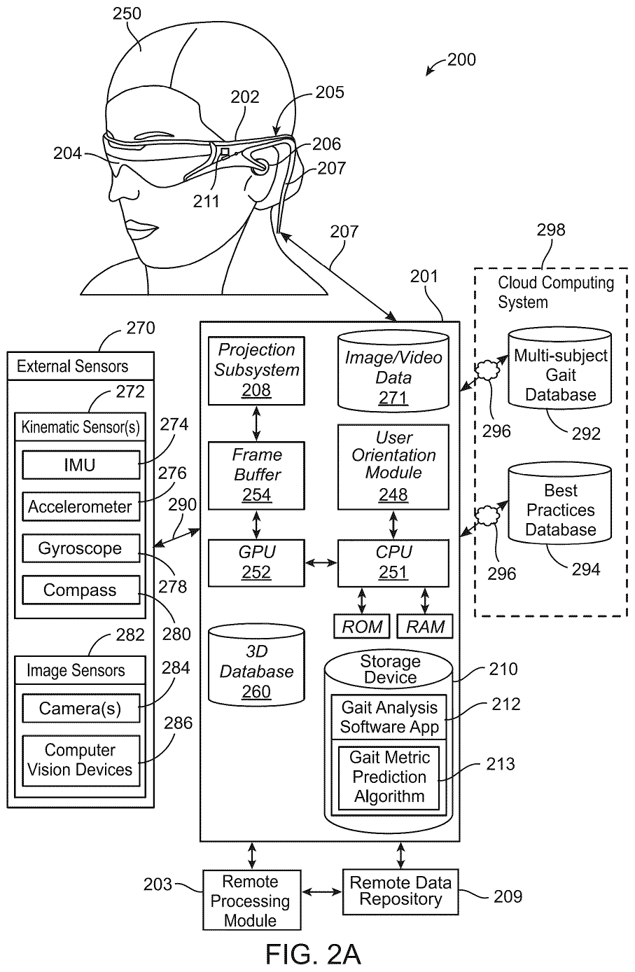

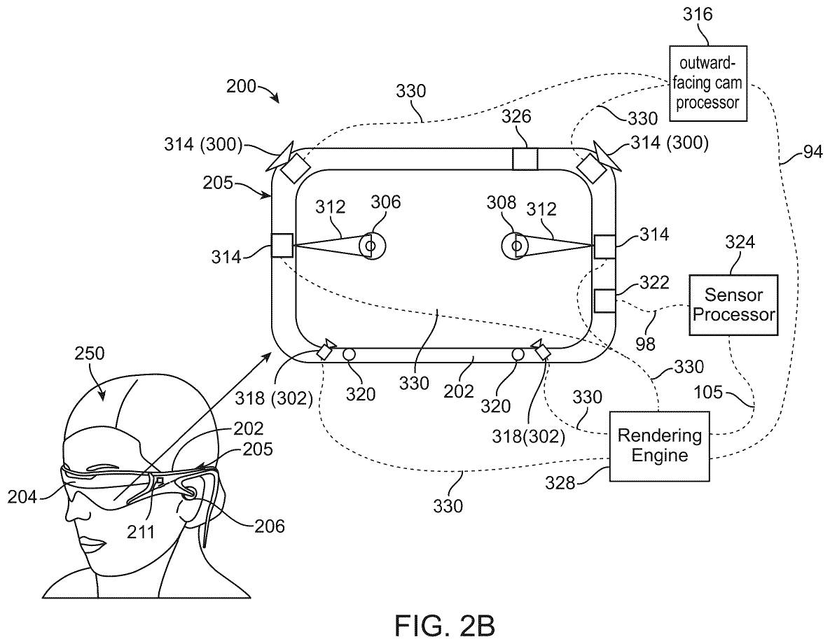

The description that follows presents an illustrative AR system 200 (see FIGS. 2A-2B) for performing gait analysis, gait disorder diagnosis, gait rehab, and feedback regarding gait rehab, as disclosed herein. However, it is to be understood that the embodiments also lend themselves to applications in other types of display systems (including other types of VR, AR, and/or MR systems), and therefore the embodiments are not to be limited to only the illustrative system disclosed herein.

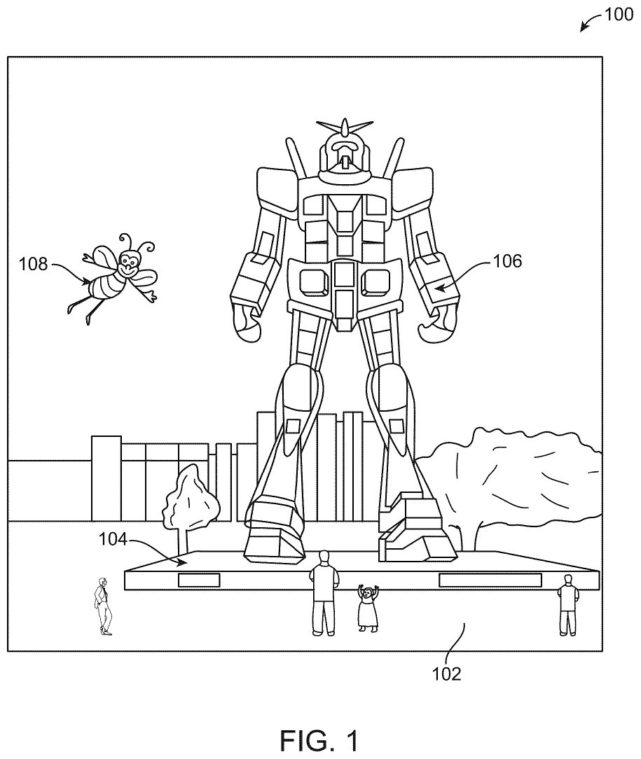

Referring to FIG. 1, AR scenarios typically include presentation of virtual content (e.g., images and sound) corresponding to virtual objects in relationship to real-world objects. For example, FIG. 1 depicts an illustration of an AR scenario with certain virtual-reality objects, and certain physical, real-world objects, as viewed by a user on a 3D display system of the AR system 200 (see FIG. 2A). As shown in FIG. 1, an AR scene 100 is depicted wherein the user of AR system 200 sees a real-world, physical, park-like setting 102 featuring people, trees, buildings in the background, and a real-world, physical concrete platform 104. In addition to these items, the user of the AR system 200 also perceives that they “see” a virtual robot statue 106 standing upon the physical concrete platform 104, and a virtual cartoon-like avatar character 108 flying by which seems to be a personification of a bumblebee, even though these virtual objects 106, 108 do not exist in the real-world.

FIGS. 2A-2B illustrate an AR system 200, according to some embodiments disclosed herein. The AR system 200 is a wearable system which comprises a display-mounted AR headset 205 which is worn on the head of the subject 250.

Referring to FIG. 2A, the AR system 200 includes a computer system 201 (also referred to as a control subsystem 201) which is operably coupled to, and in communication with, the AR headset 205. The computer system 201 includes a projection subsystem 208, providing images of virtual objects intermixed with physical objects in the AR field of view of the subject 250. This approach employs one or more at least partially transparent surfaces through which an ambient environment including the physical objects can be seen and through which the AR system 200 produces images of the virtual objects. The projection subsystem 208 is housed in the control subsystem 201 operatively coupled to a display system/subsystem 204 through a link 207. The link 207 may be a wired or wireless communication link.

In typical AR applications, various virtual objects are spatially positioned relative to respective physical objects in the field of view of the subject 250. The virtual objects may take any of a variety of forms, having any variety of data, information, concepst, or logical constructs capable of being represented as an image. Non-limiting examples of virtual objects may include a virtual target for a virtual text object, a virtual numeric object, a virtual alphanumeric object, a virtual tag object, a virtual field object, a virtual chart object, a virtual map object, a virtual instrumentation object, or a virtual visual representation of a physical object.

The AR headset 205 includes a frame structure 202 wearable by the subject 250 (e.g., wearable like a pair of eyeglasses), a 3D display system 204 carried by the frame structure 202, such that the display system 204 displays rendered 3D images into the eyes 306, 308 (see FIG. 2B) of the subject 250, and a speaker 206 incorporated into or connected to the display system 204. In the illustrated embodiment, the speaker 206 is carried by the frame structure 202, such that the speaker 206 is positioned adjacent (in or around) the ear canal of the subject 250 (e.g., an earbud, headphone, or other means of auditory display). The AR headset 205 also has one or more haptic feedback devices 211 carried by the frame structure 202 and configured to provide haptic feedback, such as vibratory tactile feedback.

The display system 204 is designed to present the eyes of the subject 250 with photo-based radiation patterns that can be comfortably perceived as augmentations to the ambient environment including both two-dimensional and three-dimensional content. The display system 204 presents a sequence of frames at high frequency that provides the perception of a single coherent scene. To this end, the display system 204 includes the projection subsystem 208 and a partially transparent display screen through which the projection subsystem 208 projects images. The display screen is positioned in a field of view of the user's 250 between the eyes of the subject 250 and the ambient environment.

In order for the 3D display to produce a true sensation of depth, and more specifically, a simulated sensation of surface depth, it may be desirable for each point in the display's visual field to generate an accommodative response corresponding to its virtual depth. If the accommodative response to a display point does not correspond to the virtual depth of that point, as determined by the binocular depth cues of convergence and stereopsis, the human eye may experience an accommodation conflict, resulting in unstable imaging, harmful eye strain, headaches, and, in the absence of accommodation information, almost a lack of surface depth.

VR, AR, and MR experiences can be provided by display systems having displays in which images corresponding to a plurality of depth planes are provided to a viewer. The images may be different for each depth plane (e.g., provide slightly different presentations of a scene or object) and may be separately focused by the viewer's eyes, thereby helping to provide the user with depth cues based on the accommodation of the eye required to bring into focus different image features for the scene located on different depth plane or based on observing different image features on different depth planes being out of focus.

As one example, to display a 3D image in an AR field of view with objects displayed such that the user perceives the objects to be in accurate locations in a world-coordinate system, in some embodiments, the projection subsystem 208 takes the form of a scan-based projection device and the display screen takes the form of a waveguide-based display into which the scanned light from the projection subsystem 208 is injected to produce, for example, images at single optical viewing distance closer than infinity (e.g., arm's length), images at multiple, discrete optical viewing distances or focal planes, and/or image layers stacked at multiple viewing distances or focal planes to represent volumetric 3D objects. These layers in the light field may be stacked closely enough together to appear continuous to the human visual subsystem (e.g., one layer is within the cone of confusion of an adjacent layer). Additionally, or alternatively, picture elements may be blended across two or more layers to increase perceived continuity of transition between layers in the light field, even if those layers are more sparsely stacked (e.g., one layer is outside the cone of confusion of an adjacent layer). The display system 204 can be monocular or binocular. The scanning assembly includes one or more light sources that produce the light beam (e.g., one that emits light of different colors in defined patterns). The light source can take any of a variety of forms, for instance, a set of RGB sources (e.g., laser diodes capable of outputting red, green, and blue light) operable to respectively produce red, green, and blue coherent collimated light according to defined pixel patterns specified in respective frames of pixel information or data. Laser light provides high color saturation and is highly energy efficient. The optical coupling subsystem includes an optical waveguide input apparatus, such as, for instance, one or more reflective surfaces, diffraction gratings, mirrors, dichroic mirrors, or prisms to optically couple light into the end of the display screen. The optical coupling subsystem further includes a collimation element that collimates light from the optical fiber. Optionally, the optical-coupling subsystem includes an optical-modulation apparatus configured for converging the light from the collimation element toward a focal point in the center of the optica-waveguide input apparatus, thereby allowing the size of the optical waveguide input apparatus to be minimized. Thus, the display system 204 generates a series of synthetic image frames of pixel information that present an undistorted image of one or more virtual objects to the user. Further details describing display subsystems are provided in U.S. Utility patent application Ser. No. 14/212,961, entitled “Display System and Method” (Attorney Docket No. ML.20006.00), and Ser. No. 14/331,218, entitled “Planar Waveguide Apparatus With Diffraction Element(s) and Subsystem Employing Same” (Attorney Docket No. ML.20020.00), the contents of which are hereby expressly and fully incorporated by reference in their entirety, as though set forth in full.

The AR system 200 further includes one or more sensors mounted to the frame structure 202, some of which are described herein with respect to FIG. 2B, for detecting the position (including orientation) and movement of the head of the subject 250 and/or the eye position and interocular distance of the subject 250. Such sensor(s) may include image capture devices (e.g., cameras 318 in an inward-facing imaging system and/or cameras 314 in an outward-facing imaging system), audio sensor (e.g., microphones), GPS units, radio devices, and kinematic sensors such as inertial measurement units (IMUs), accelerometers, compasses, gyros, and the like. For example, in one embodiment, the AR system 200 includes a head worn transducer subsystem that includes one or more inertial transducers to capture inertial measures indicative of movement of the head of the subject 250. Such devices may be used to sense, measure, or collect information about the head movements of the subject 250. For instance, these devices may be used to detect/measure movements, speeds, acceleration and/or positions of the head of the subject 250. The position (including orientation) of the head of the subject 250 is also known as a “head pose” of the subject 250.

The AR system 200 of FIG. 2A includes an outward-facing imaging system 300 (see FIG. 2B) which for capturing images of the surrounding environment around the subject 250. The outward-facing imaging system 300 comprises one or more outward-facing cameras 314 configured to capture images of the surrounding environment and provide image data to the computer system 201. The cameras 314 include cameras facing in outward directions from the subject 250, including the front, rear and sides of the subject 250, and above and/or below the subject 250. The outward-facing imaging system 300 can be employed for any number of purposes, such as detecting and tracking objects around the user, recording of images/video of the environment surrounding the subject 250, and/or capturing information about the environment in which the subject 250 is located, such as information indicative of distance, orientation, and/or angular position of the subject 250 and objects around the subject 250 with respect to the environment around the subject 250.

The AR system 200 may further include an inward-facing imaging system 304 (see FIG. 2B) which can track the angular position (the direction in which the eye or eyes are pointing), movement, blinking, and/or depth of focus (by detecting eye convergence) of the eyes 306, 308 of the subject 250. Such eye tracking information may, for example, be discerned by projecting light at the user's eyes, 306, 308, and detecting the return or reflection of at least some of that projected light.

The control subsystem 201 can take any of a variety of forms. The control subsystem 201 includes a number of controllers, for instance one or more microcontrollers, microprocessors or central processing units (CPUs), digital signal processors, graphics processing units (GPUs), other integrated circuit controllers, such as application specific integrated circuits (ASICs), programmable gate arrays (PGAs), for instance field PGAs (FPGAs), and/or programmable logic controllers (PLUS). The control subsystem 201 includes a digital signal processor (DSP), one or more central processing units (CPUs) 251, one or more graphics processing units (GPUs) 252, and one or more frame buffers 254. The CPU 251 controls overall operation of the AR system 200, while the GPU 252 renders frames (i.e., translating a three-dimensional scene into a two-dimensional image) and stores these frames in the frame buffer(s) 254. While not illustrated, one or more additional integrated circuits may control the reading into and/or reading out of frames from the frame buffer(s) 254 and operation of the display system 204. Reading into and/or out of the frame buffer(s) 254 may employ dynamic addressing, for instance, where frames are over-rendered. The control subsystem 201 further includes a read only memory (ROM) and a random access memory (RAM). The control subsystem 201 further includes a three-dimensional database 260 from which the GPU 252 can access three-dimensional data of one or more scenes for rendering frames, as well as synthetic sound data associated with virtual sound sources contained within the three-dimensional scenes.

The control subsystem 201 can also include an image/video database 271 for storing the image/video and other data captured by the outward-facing imaging system 300, the inward-facing imaging system 302, and/or any other camera(s) and/or sensors of the AR system 200.

The control subsystem 201 can also include a user orientation detection module 248. The user orientation module 248 detects an instantaneous position of the head of the subject 250 and may predict a position of the head of the subject 250 based on position data received from the sensor(s). The user orientation module 248 also tracks the eyes of the subject 250, and in particular the direction and/or distance at which the subject 250 is focused based on the tracking data received from the sensor(s).

The various processing components of the AR systems 200 may be contained in a distributed subsystem. For example, the AR system 200 may include a local processing and data module (i.e., the control subsystem 201) operatively coupled, such as by a wired lead or wireless connectivity 207, to a portion of the display system 204. The local processing and data module may be mounted in a variety of configurations, such as fixedly attached to the frame structure 202, fixedly attached to a helmet or hat, embedded in headphones, removably attached to the torso of the subject 250, or removably attached to the hip of the subject 250 in a belt-coupling style configuration. The AR system 200 may further include a remote processing module 203 and remote data repository 209 operatively coupled, such as by a wired lead or wireless connectivity to the local processing and data module 203, such that these remote modules are operatively coupled to each other and available as resources to the local processing and data module 203. The local processing and data module 201 may comprise a power-efficient processor or controller, as well as digital memory, such as flash memory, both of which may be utilized to assist in the processing, caching, and storage of data captured from the sensors and/or acquired and/or processed using the remote processing module 203 and/or remote data repository 209, possibly for passage to the display system 204 after such processing or retrieval. The remote processing module 203 may comprise one or more relatively powerful processors or controllers configured to analyze and process data and/or image information. The remote data repository 209 may comprise a relatively large-scale digital data storage facility, which may be available through the Internet or other networking configuration in a “cloud” resource configuration. In some embodiments, all data is stored and all computation is performed in the local processing and data module 201, allowing fully autonomous use from any remote modules. The couplings between the various components described above may include one or more wired interfaces or ports for providing wires or optical communications, or one or more wireless interfaces or ports, such as via RF, microwave, and IR for providing wireless communications. In some implementations, all communications may be wired, while in other implementations all communications may be wireless, with the exception of the optical fiber(s).

The AR system 200 also includes a storage device 210 for storing software applications to program the AR system 200 to perform application specific functions. The storage device 210 which may be any suitable storage device such as a disk drive, hard drive, solid state drive (SSD), tape drive, etc. The storage device 210 for storing software applications may also be any one of the other storage devices of the AR system, and is not required to be a separate, stand-alone storage device for software applications. The storage device 210 comprises a non-transitory computer readable medium. Common forms of non-transitory computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

For example, the 3D database 260, and/or image/video data 271 may be stored on the same storage device. A gait-analysis software application 212 is stored on the storage device 210. The gait-analysis software application 212 includes a gait-metric prediction algorithm 213.

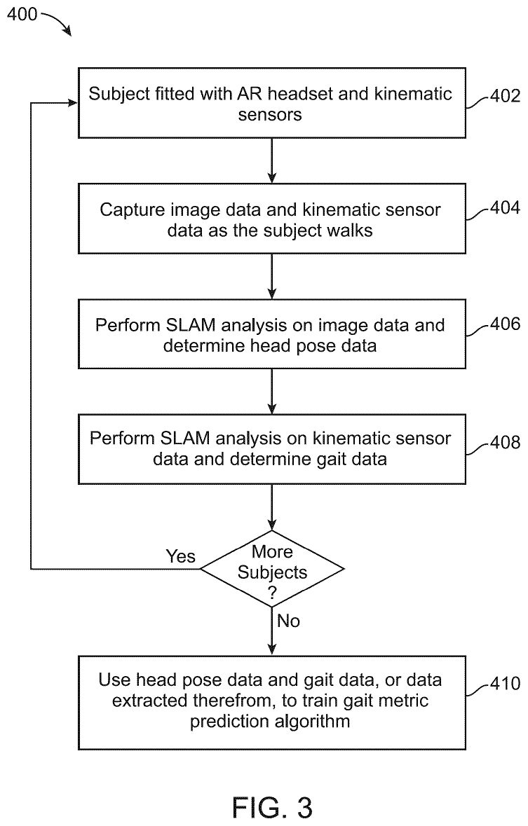

The gait-metric prediction algorithm 213 is a trained model using a deep learning approach. FIG. 3 is a flow chart illustrating one embodiment of a method 400 for developing the gait-metric prediction algorithm. 213 The training data is obtained for a plurality of subjects 250 in which the training data can be used to correlate respective head-pose data with respective gait data for each subject. At step 402, a subject is fitted with the AR headset 205 and a plurality of kinematic sensors 272 (see FIG. 2A) worn on different parts of the subject's body (e.g., motion sensors on the feet, legs, hips, etc.) for obtaining motion data of the different parts of the subject's body. Then, at step 404, image data from the cameras 314 on the AR headset 205 and kinematic sensor data from the kinematic sensors 272 is captured as the subject 250 walks. At step 406, a SLAM analysis is performed on the image data to determine time-dependent (e.g., time-stamped) head-pose data. At step 408, a SLAM analysis is performed on the kinematic sensor data to determine time-dependent (e.g., time-stamped) gait data, such as motion data for the different parts of the subject's body. This process is repeated for the desired number of subjects 250 in the plurality of subjects 250. At step 410, the head-pose data and gait data, and/or data extracted from the head-pose data and gait data (e.g., the head-pose data and gait data may undergo post-processing), are then used as training data to train the gait-metric prediction algorithm 213. In method of post-processing the gait data, the gait data may be further processed and/or analyzed to extract gait attributes, which are used as part of the training data. As some non-limiting examples, the trained-model can utilize a convolutional neural network (CNN) or a long term short term memory (LSTM) neural network, or other suitable deep learning technique.

The AR system 200 may also include one or more external sensors 270 which are external to, and not carried by, the AR headset 205. The external sensors 270 may comprise one or more kinematic sensors 272, such as an IMU 274, one or more accelerometer(s) 276, one or more gyroscopes 278, and one or more compasses or other directional devices 280. The external sensors 270 may also include one or more external image sensor(s) 282 which may be camera(s) 284 or computer vision device(s) 286. Each of the external sensors 270 is operably coupled to, and in communication with the computer system 201 via a communication link 290, which may be a wired or wireless communication link. Each of the external sensors 270 may be operably coupled to a sensor processor, such as a sensor processor 324 of the computer system 201, as described herein. The sensor processor is configured to execute digital or analog processing of the sensor data (e.g., image data for the image sensor(s) 282, kinematic sensor data for the kinematic sensor(s) 272, etc.) received from the respective external sensor 270.

Turning to FIG. 2B, the AR system 200 is shown along with an enlarged schematic view of the headset 205 and various components of the headset 205. In certain implements, one or more of the components illustrated in FIG. 2B can be part of the 3D display system 204. The various components alone or in combination can collect a variety of data (such as e.g., audio or visual data) associated with the subject 250 of the wearable system 200 or the subject's environment. It should be appreciated that other embodiments may have additional or fewer components depending on the application for which the wearable system is used. Nevertheless, FIG. 2B illustrates one exemplary embodiment of the AR system 200 for performing motor skills neurological tests as described herein.

As shown in FIG. 2B, the AR system 200 includes the 3D display system 204. The display system 204 comprises a display lens 310 that is on the wearable frame 202. The display lens 310 may comprise one or more transparent mirrors positioned by the frame 220 in front of the user's eyes 306, 308 and may be configured to bounce projected light beams 312 comprising the AR images into the user's eyes 306, 308 and facilitate beam shaping, while also allowing for transmission of at least some light from the environment around the subject 250. The wavefront of the projected light beams 312 may be bent or focused to coincide with a desired focal distance of the projected light.

Outward-facing cameras 314, which are part of the outward-facing imaging system 300, are mounted on the frame 220 and are directed outward from the subject 250 to capture images (the term “image” as used herein also includes video) of the surrounding environment. The cameras 314 may be two wide-field-of-view machine vision cameras 314 (also referred to as world cameras), or any other suitable cameras or sensors. For instance, the cameras 314 may be dual capture visible light/non-visible (e.g., infrared) light cameras. Images acquired by the cameras 314 are processed by an outward-facing imaging processor 36. The outward-facing imaging processor 316 implements one or more image processing implements one or more image processing applications to analyze and extract data from the images captured by the cameras 314. The outward-facing imaging processor 316 includes an object recognition application which implements an object recognition algorithm to recognize objects within the images, including recognizing various body parts of the user, including a user's hands, fingers, arms, legs, etc. The outward-facing imaging processor 316 also includes an object tracking application which implements an object tracking algorithm which tracks the location and movement of an object registered to a world coordinate system common to the 3D virtual location of virtual objects displayed to the subject 250 on the 3D display 220. In other words, the tracked location of the real objects in the real world is relative to the same world coordinate system as the virtual images in an AR field of view displayed on the 3D display 220. The outward-facing imaging processor 316 may also include a pose processing application which implements a pose detection algorithm which identifies a pose of the subject 250, i.e., the location and head/body position of the subject 250. The outward-facing imaging processor 316 may be implemented on any suitable hardware, such as an ASIC (application specific integrated circuit), FPGA (field programmable gate array), ARM processor (advanced reduced-instruction-set machine), or as part of the control subsystem 201. The outward-facing imaging processor 316 may be configured to calculate real or near-real time pose, location and/or tracking data using the image information output from the cameras 314.

With continued reference to FIG. 2B, the headset 205 also includes a pair of scanned-laser shaped-wavefront (e.g., for depth) light projector modules 314 having display mirrors and optics configured to project the light 312 into the user's eyes 306, 308. The headset 205 also has inward-facing cameras/sensors 318, which are part of the inward-facing imaging system 302, mounted on the interior of the frame 220 and directed at the user's eyes 306, 308. The cameras 318 may be two miniature infrared cameras 318 paired with infrared light sources 320 (such as light emitting diodes “LED”s), which are configured to track the gaze of the user's eyes 306, 308 user to support rendering of AR images, for user input (e.g., gaze activated selection of user inputs), and also to determine a correlation between a proficiency of the user's eye tracking and the quality of movement of the user's body part from a starting location to a target location, as discussed in more detail herein. The user's eye tracking data can be used to evaluate the smoothness of the user's eye tracking during the test, and can enable more comprehensive clinical evaluation of the patient's motor skills function. Furthermore, the AR system 200 is configured to determine a correlation between the proficiency of the user's eye tracking and the quality of movement of the body part from the starting location to the target location. This correlation data representative of the correlation between a proficiency of the user's eye tracking and the quality of movement of the body part from the starting location to the target location can be provided to the clinician. The correlation data can then be used by a clinician to further evaluate and diagnose the user's condition.

The AR system 200 may also have a sensor assembly 322, which may comprise an X, Y, and Z axis accelerometer capability as well as a magnetic compass and X, Y, and Z axis gyro capability, preferably providing data at a relatively high frequency, such as 200 Hz. The sensor assembly 322 may comprise, or be part of, the IMU described with reference to FIG. 2A.

Still referring to FIG. 2B, the AR system 200 may also include a sensor processor 324 configured to execute digital or analog processing of the data received from the gyro, compass, and/or accelerometer of the sensor assembly 322. The sensor processor 324 may be part of the local control subsystem 201 shown in FIG. 2A. The AR system 200 may also include a position system 326 such as, e.g., a GPS module 326 (global positioning system) to assist with pose and positioning analyses. In addition, the GPS 326 may further provide remotely-based (e.g., cloud-based) information about the user's environment. This information may be used for recognizing objects or information in user's environment.

The AR system 200 may combine data acquired by the GPS 326 and a remote computing system (such as, e.g., the remote processing module 203) which can provide more information about the user's environment. As one example, the wearable system can determine the user's location based on GPS data and retrieve a world map (e.g., by communicating with a remote processing module 203) including virtual objects associated with the user's location. As another example, the wearable system 200 can monitor the environment using the cameras 314. Based on the images acquired by the outward-facing cameras 314, the wearable system 200 can detect characters in the environment (e.g., by using the object recognition application of the outward-facing imaging processor 316). The AR system 200 can further use data acquired by the GPS 326 to interpret the characters. For example, the AR system 200 can identify a geographic region where the characters are located and identify one or more languages associated with the geographic region. The AR system 200 can accordingly interpret the characters based on the identified language(s), e.g., based on syntax, grammar, sentence structure, spelling, punctuation, etc., associated with the identified language(s). In one example, a user in Germany can perceive a traffic sign while driving down the autobahn. The AR system 200 can identify that the user is in Germany and that the text from the imaged traffic sign is likely in German based on data acquired from the GPS 326 (alone or in combination with images acquired by the cameras 314).

In some situations, the images acquired by the cameras 314 may include incomplete information of an object in a user's environment. For example, the image may include an incomplete text (e.g., a sentence, a letter, or a phrase) due to a hazy atmosphere, a blemish or error in the text, low lighting, fuzzy images, occlusion, limited FOV of the cameras 314 etc. The AR system 200 could use data acquired by the GPS 326 as a context clue in recognizing the text in image.

The AR system 200 may also comprise a rendering engine 328 which can be configured to provide rendering information that is local to the subject 250 to facilitate operation of the scanners and imaging into the eyes 306, 308 of the subject 250, for the user's view of the world. The rendering engine 328 may be implemented by a hardware processor (such as, e.g., a central processing unit or a graphics processing unit). In some embodiments, the rendering engine 328 is part of the control subsystem 201.

The components of the AR system 200 are communicatively coupled to each other via one or more communication links 330. The communication links may be wired or wireless links, and may utilize any suitable communication protocol. For example, the rendering engine 328, can be operably coupled to the cameras 318 via communication link 330, and be coupled to the projection subsystem 208 (which can project light 312 into user's eyes 306, 308 via a scanned laser arrangement in a manner similar to a retinal scanning display) via the communication link 330. The rendering engine 328 can also be in communication with other processing units such as, e.g., the sensor processor 324 and the outward-facing camera processor 316 via links 330.

The cameras 318 (e.g., mini infrared cameras) may be utilized to track the eye pose to support rendering and user input. Some examples of eye poses include where the user is looking or at what depth he or she is focusing (which may be estimated with eye vergence). The GPS 326, gyros, compass, and accelerometers 322 may be utilized to provide coarse or fast pose estimates. One or more of the cameras 314 can also acquire images and pose data, which in conjunction with data from an associated cloud computing resource, may be utilized to map the local environment and share user views with others.

The example components depicted in FIG. 2B are for illustration purposes only. Multiple sensors and other functional modules are shown together for ease of illustration and description. Some embodiments may include only one or a subset of these sensors or modules. Further, the locations of these components are not limited to the positions depicted in FIG. 2B. Some components may be mounted to or housed within other components, such as a belt-mounted component, a hand-held component, or a helmet component. As one example, the outward-facing camera processor 316, sensor processor 324, and/or rendering engine 328 may be positioned in a belt-pack and configured to communicate with other components of the AR system 200 via wireless communication, such as ultra-wideband, Wi-Fi, Bluetooth, etc., or via wired communication. The depicted frame 2015 may be head-mountable and wearable by the subject 250. However, some components of the AR system 200 may be worn on other portions of the user's body. For example, the speaker 206 may be inserted into the ears of the subject 250 to provide sound to the subject 250.

Regarding the projection of light 312 into the eyes 306, 308 of the subject 250, in some embodiment, the cameras 318 may be utilized to measure where the centers of a user's eyes 306, 308 are geometrically verged to, which, in general, coincides with a position of focus, or “depth of focus”, of the eyes 306, 308. A 3-dimensional surface of all points the eyes verge to can be referred to as the “horopter”. The focal distance may take on a finite number of depths, or may be infinitely varying. Light projected from the vergence distance appears to be focused to the subject eye 306, 308, while light in front of or behind the vergence distance is blurred. Examples of wearable devices and other display systems of the present disclosure are also described in U.S. Patent Publication No. 2016/0270656, which is incorporated by reference herein in its entirety.

Further spatially coherent light with a beam diameter of less than about 0.7 millimeters can be correctly resolved by the human eye regardless of where the eye focuses. Thus, to create an illusion of proper focal depth, the eye vergence may be tracked with the cameras 24, and the rendering engine 34 and projection subsystem 18 may be utilized to render all objects on or close to the horopter in focus, and all other objects at varying degrees of defocus (e.g., using intentionally-created blurring). Preferably, the system 220 renders to the user at a frame rate of about 60 frames per second or greater. As described above, preferably, the cameras 24 may be utilized for eye tracking, and software may be configured to pick up not only vergence geometry but also focus location cues to serve as user inputs. Preferably, such a display system is configured with brightness and contrast suitable for day or night use.

In some embodiments, the display system preferably has latency of less than about 20 milliseconds for visual object alignment, less than about 0.1 degree of angular alignment, and about 1 arc minute of resolution, which, without being limited by theory, is believed to be approximately the limit of the human eye. The display system 204 may be integrated with a localization system, which may involve GPS elements, optical tracking, compass, accelerometers, or other data sources, to assist with position and pose determination; localization information may be utilized to facilitate accurate rendering in the user's view of the pertinent world (e.g., such information would facilitate the glasses to know where they are with respect to the real world).

The AR system 200 is programmed by the gait-analysis software application 212 to perform gait analysis, gait disorder diagnosis, gait rehab functions, and feedback regarding gait rehab, as disclosed herein.

The AR system 200 may also be in communication with a multi-subject gait database 292 and a best practices database 294 via a communication network 296. The multi-subject gait database 292 and best practices database 294 may be implemented on a cloud computing system 298 (i.e., cloud computing resources), or on private computing resources. The multi-subject gait database 292 includes one or more storage devices which store respective gait information for a plurality of respective subjects 250. The gait information can include subject identification information, gait attributes, gait diagnosis, gait rehab treatment, gait rehab progress, etc. for each subject 250. As the AR system 200 generates gait information for each subject, the gait information is securely transmitted to, and stored on, the multi-subject gait database 292. The gait information can include subject identification information, gait attributes, gait diagnosis, gait rehab treatment, gait rehab progress, etc. for each subject 250. The AR system 200 can also securely access the gait information for each subject via log-in credentials from the AR system 200. The best practices database 294 one or more storage devices which store best practices information for diagnosing gait disorders and/or respective rehab treatment and rehab tasks for respective gait disorders or gait attributes. The AR system 200 utilizes the best practices database 294 in order to analyze gait attributes to diagnose a gait disorder and/or determine a rehab treatment and rehab tasks to prescribe to treat the subject's gait disorder.

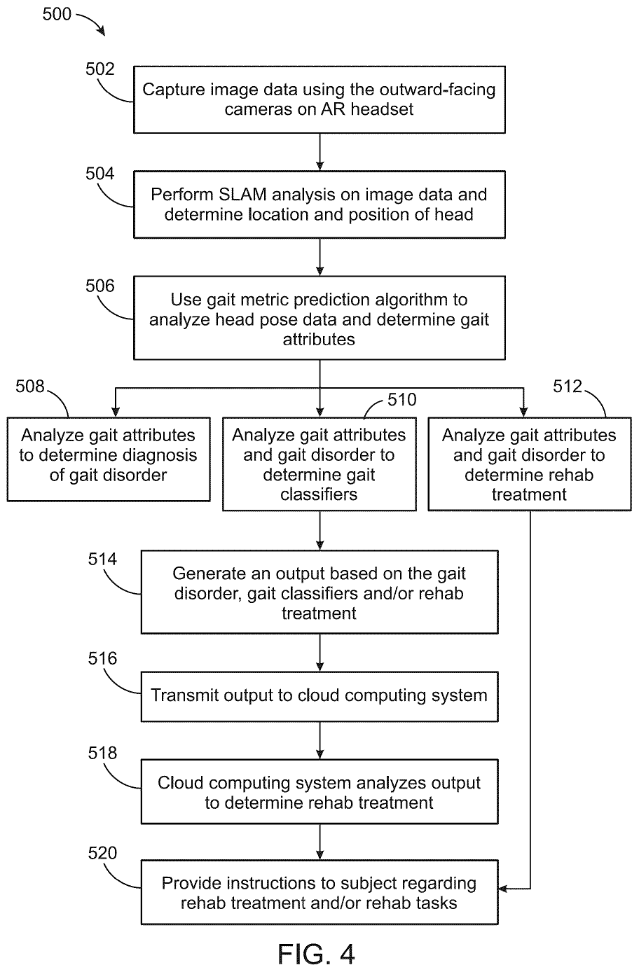

Turning now to FIG. 4, a flow chart shows one embodiment of a method 500 for performing a gait analysis using the AR system 200 as programmed by the gait-analysis software application 212. With the subject 250 wearing the AR headset 205, at step 502 of the method 500, the outward-facing cameras 314 on the AR headset 205 capture image data of the environment surrounding the subject as the subject 250 walks. At step 504, the computer system 201 performs a SLAM analysis on the image data and determines head-pose data regarding the location in three-dimensional coordinate space (e.g., X, Y, Z coordinates) and position (e.g., orientation, fore/aft tilt, left/right tilt and/or left/right rotation) of the subject's head as the subject 250 walks. Thus, the head-pose data includes data representative of the time-dependent, three-dimensional coordinate location of the head, and the fore/aft tilt position, left/right tilt position, and/or left/right rotational position of the head.

At step 506, the computing system 201 uses the gait-metric prediction algorithm 213 to analyze the head-pose data and to determine one or more gait attributes of the subject which characterize the subject's gait. The gait attributes can include such metrics as step length, step width, step velocity, step quantity, step cadence (steps/unit of time), stance time, gait velocity, gait symmetry, foot pressure, other musculoskeletal kinematic features, and/or any other suitable metrics. At step 508, the method 500 may further include analyzing the one or more gait attributes to determine a diagnosis of a gait disorder of the subject. As some examples of gait disorders, step 508 may determine that the subject has a limp or an asymmetrical gait caused by a stroke, injury, orthopedic surgery, etc. At step 510, the computing system 201 analyzes the one or more gait attributes to determine one or more gait classifiers based on the one or more gait attributes. The gait classifiers may include classifiers such as a gait disorder caused by a neurodegenerative disease, a neurological trauma, a musculoskeletal injury, an orthopedic surgery, or the like. At step 512, computing system 201 analyzes the one or more gait attributes, and optionally the determined gait disorder, to determine a course of rehabilitation treatment for treating the gait disorder. The computing system 201 may access and utilize data and/or analysis algorithms stored on the best practices database 294 to determine the rehabilitation treatment. As non-limiting examples, the treatment may be a course of exercise, physical therapy, or the like, including the exercises, frequency (e.g., once a day, twice a week, etc.), duration of each session (30 minutes, 1 hour, etc.), and length of treatment (e.g., 3 months, 6 months, 1 year, until predetermined level of improvement in certain gait attribute(s), etc.).

At step 514, the computing system 201 generates an output based on the gait attributes, gait disorder, gait classifiers and/or rehab treatment. For example, the output may be a data package for the cloud computing system to use in determining a course of rehabilitation treatment, alternative to the computing system 201 making such determination. To that end, at step 516, the computing system 201 transmits the output to the cloud computing system 298 via the communication network 296, and at step 518, the cloud computing system 298, utilizing the best practices database 294, analyzes the output and determines a course of rehabilitation treatment. The cloud computing system 298 may then provide the course of rehabilitation treatment via the communication network 296 to the computing system 298, or to a health care provider for providing the rehab treatment to the subject.

At step 520, instructions are provided to the subject regarding rehabilitation tasks for the course of rehabilitation treatment to treat the gait disorder. The instructions may be provided to the subject in various ways. In one embodiment, AR system 200 presents rehabilitation task virtual content regarding the rehabilitation task to the subject via the AR headset 205. The rehabilitation task virtual content comprises video instructions for performing the rehabilitation task which are presented via the 3D display system 204, the speaker 206, and haptic devices 211 of the AR headset 205. As one example, the rehab task virtual content may include a game-engine based synthetic visual representation of a physical therapist that interacts with the subject 250 via synthetic speech, visual gestures, audio and haptic feedback. Alternatively, the rehab task virtual content may be in the form of live streamed virtual video of a clinician providing instructions to the subject 250 for performing the rehabilitation task presented on the AR headset 205.

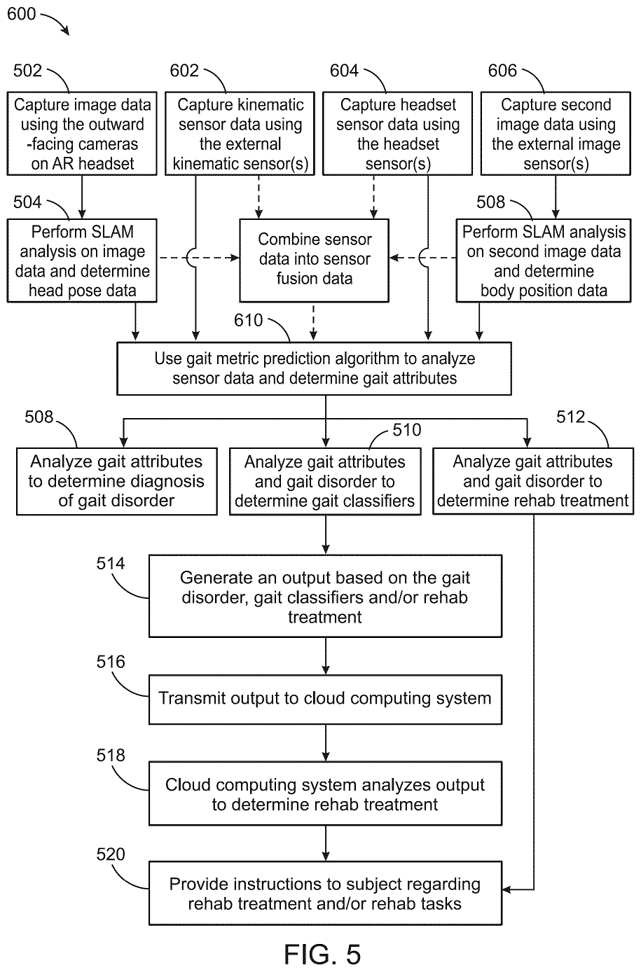

Referring now to FIG. 5, a flow chart shows another embodiment of a method 600 for performing a gait analysis using the AR system 200 as programmed by the gait-analysis software application 212. The method 600 is similar to the method 500 except that method 600 utilizes the other sensors in addition to the outward-facing cameras 314 in order to determine the gait attributes. Thus, the steps in method 600 are the same as the same numbered steps in the method 500, and the description above for such steps in method 500 applies equally to method 600. The method 600 may utilize any one or more of the external sensors 270 worn on different parts of the body other than the head, including the kinematic sensors 272 (IMU 274, accelerometer 276, gyroscope 278, compass or other directional device 278), and/or external image sensors 282 (which may be worn on the subject 250 and/or positioned around the subject 250), and/or any one or more of the other headset sensors carried by the AR headset 205, including the sensors of the sensor assembly 322 (including the IMU, gyro, compass, and/or accelerometer of the sensor assembly 322). Accordingly, in method 600, at step 602, the kinematic sensors 272 obtain kinematic sensor data as the subject 250 walks, at the same time as step 502 in which the outward-facing cameras 314 on the AR headset 205 are capturing image data. Also, at the same time as step 502, at step 604, the headset sensors obtain headset sensor data as the subject 250 walks, and at step 606, the external image sensors 282 capture second image data. At step 608, the computer system 201 performs a SLAM analysis on the second image data and determines body position data regarding the time-dependent location in three-dimensional coordinate space (e.g., X, Y, Z coordinates) and position (e.g., orientation, fore/aft tilt, left/right tilt and/or left/right rotation) of the one or more body parts of the subject 250 (e.g., legs, feet, hips, etc.) subject's head as the subject 250 walks. This is similar to the SLAM analysis in step 504, as described herein.

At step 610, two or more of the head-pose data, kinematic sensor data, headset sensor data and/or body position data may be combined into sensor fusion data. The various data can be combined using a fusion formula which applies different weights to the head-pose data, kinematic sensor data, headset sensor data and/or body position data to determine the sensor fusion data. Step 610 is an optional step, and is not required in the method 600.

At step 612, the computing system 201 uses the gait-metric prediction algorithm 213 to analyze one or more of the head-pose data, kinematic sensor data, headset sensor data, body position data, and/or sensor fusion data and to determine one or more gait attributes of the subject which characterize the subject's gait. Any combination of the head-pose data, kinematic sensor data, headset sensor data, body position data, and/or sensor fusion data may be utilized. As just one example of the many possible combinations, the gait attributes may be determined using a combination of the head-pose data, body position data, and a combination of the headset sensor data and kinematic sensor data.

The method 600 then includes the same steps 508-520 as described above with respect to method 500.

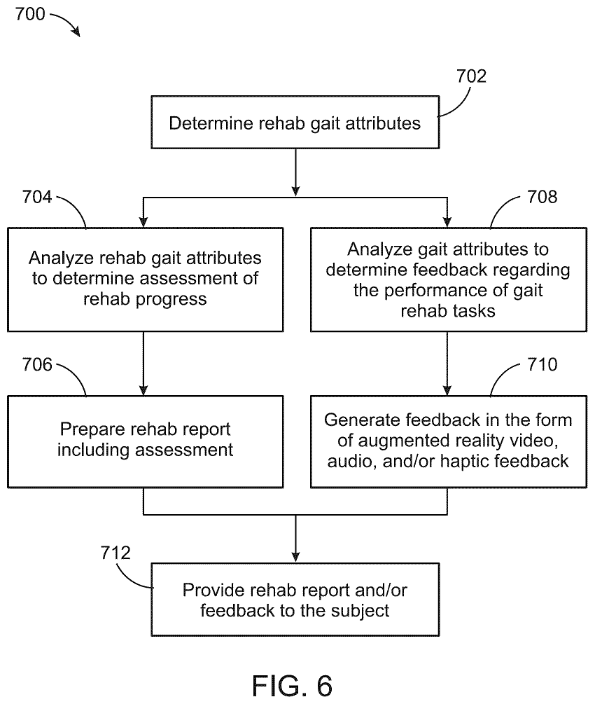

Referring now to FIG. 6, a flow chart illustrates another embodiment of a method 700 disclosed herein for using the AR system 200 as programmed by the gait-analysis software application 212 to analyze a subject performing gait rehabilitation (rehab) tasks, assess the subject's progress, and/or provide feedback to the subject. At step 702 of method 700, the AR system 200 determines rehab gait attributes of the subject 250 performing one or more rehab tasks. The AR system 200 may determine these gait attributes using any suitable method, such as method 600 (e.g., steps 502-506 of method 500) or method 600 (e.g., steps up to step 610). For instance, when using method 500, the step 502 captures rehab image data from the one or more image sensors 314 on the AR headset 205 of the subject 250 performing a gait rehabilitation task for treating a gait disorder (e.g., the gait rehab task determined by step 512 or step 518). At step 504 of method 500, the computer system 201 performs a SLAM analysis on the rehab image data to determine rehab head-pose data of the head of the subject 250, same or similar to the SLAM analysis on the original image data. Then, at step 506, the rehab head-pose data is analyzed using the gait-metric prediction algorithm 213 to determine one or more rehab gait attributes/metrics of the gait of the subject 250 during the rehab tasks. Similarly, when using method 600 to determine rehab gait attributes, the method obtains rehab sensor data and rehab image data, and processes the data to determine rehab head-pose data, rehab body position data, rehab sensor fusion data, and analyzes such data using the gait-metric prediction algorithm 213 to determine the rehab gait attributes

At step 704, the AR system 200 analyzes the rehab gait attributes to determine an assessment of the subject's rehab progress. In one way, the AR system 200 can access the multi-subject gait database 292 and compare the current rehab gait attributes to previous gait attributes obtained from previous rehab sessions for the subject and/or from other subjects in the database 292. At step 706, The AR system 200 prepares a rehab report including an assessment of the subject's rehab progress.

At step 708, the AR system 200 analyzes the rehab gait attributes to determine feedback regarding the subject's performance of the gait rehab tasks. For example, the AR system 200 analyzes the rehab gait attributes and determines if the subject is correctly performing the rehab tasks, and if not, the AR system 200 generates feedback to advise the subject of the errors and/or instructions for making corrections in performing the rehab tasks. At step 710, the AR system 200 generates feedback to provide to the subject 250. At step 712, the AR system 20 provides the rehab report and/or feedback to the subject. For example, the report and feedback may be provided to the subject via the AR headset 205, including augmented reality video, audio and/or feedback via the haptic feedback devices 211.

The disclosure includes methods that may be performed using the disclosed systems and devices. The methods may comprise the act of providing such suitable systems and devices. Such provision may be performed by the user. In other words, the “providing” act merely requires the user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the disclosure, together with details regarding material selection and manufacture have been set forth above. As for other details of the present disclosure, these may be appreciated in connection with the above-referenced patents and publications as well as generally known or appreciated by those with skill in the art. The same may hold true with respect to method-based aspects of the disclosure in terms of additional acts as commonly or logically employed.

In addition, though the disclosure has been described in reference to several examples optionally incorporating various features, the disclosure is not to be limited to that which is described or indicated as contemplated with respect to each variation of the disclosure. Various changes may be made to the disclosure described and equivalents (whether recited herein or not included for the sake of some brevity) may be substituted without departing from the true spirit and scope of the disclosure. In addition, where a range of values is provided, it is understood that every intervening value, between the upper and lower limit of that range and any other stated or intervening value in that stated range, is encompassed within the disclosure.

Also, it is contemplated that any feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present disclosure is not to be limited to the examples provided and/or the subject specification, but rather only by the scope of claim language associated with this disclosure.

In the foregoing specification, the disclosure has been described with reference to specific embodiments thereof. It will, however, be evident that various modifications and changes may be made thereto without departing from the broader spirit and scope of the disclosure. For example, the above-described process flows are described with reference to a particular ordering of process actions. However, the ordering of many of the described process actions may be changed without affecting the scope or operation of the disclosure. The specification and drawings are, accordingly, to be regarded in an illustrative rather than restrictive sense.

1. A computer-implemented method for performing a gait analysis using an AR headset worn on the head of a subject, the AR headset having a frame structure configured to be worn on the head and a display disposed on the frame structure, the method comprising:
capturing image data from one or more image sensors disposed on the AR headset as the subject walks;
performing a SLAM analysis on the image data to determine head-pose data regarding a position and location of the head of the subject; and
determining one or more gait attributes of a gait of the subject by analyzing the head-pose data using a gait-metric prediction algorithm.

2. The method of claim 1, wherein the one or more gait attributes comprises at least one of a step length, a step width, a step velocity, a step quantity, a step cadence, a stance time, a gait velocity, a gait symmetry, foot pressure and musculoskeletal kinematic features.

3. The method of claim 1, wherein the head-pose data includes data representative of a time-dependent, three-dimensional coordinate location of the head.

4. The method of claim 3, wherein the head-pose data further includes a fore/aft tilt position of the head and a left/right rotational position of the head.

5. The method of claim 1, further comprising:
analyzing the one or more gait attributes to determine a diagnosis of a gait disorder of the subject.

6. The method of claim 5, further comprising:
analyzing the one or more gait attributes to determine a course of rehabilitation treatment for treating the gait disorder.

7. The method of claim 1, further comprising:
analyzing the one or more gait attributes to determine a rehabilitation treatment for treating a gait disorder of the subject.

8. The method of claim 1, further comprising:
obtaining second sensor data from a second sensor different from the one or more image sensors; and
wherein the step of determining the one or more gait attributes comprises analyzing the head-pose data and the second sensor data using the gait-metric prediction algorithm.

9. The method of claim 8, wherein the head-pose data and the second sensor data is combined prior to analyzing the head-pose data and second sensor data using the gait-metric prediction algorithm.

10. The method of claim 9, wherein the head-pose data and the second sensor data is combined using a fusion formula which applies different weights to head-pose data and the second sensor data.

11. The method of claim 8, wherein the second sensor is a kinematic sensor comprising one or more of an inertial measurement unit, an accelerometer, a direction sensor, a compass, a gyroscope, a camera, and a computer vision sensor.

12. The method of claim 8, wherein the second sensor is worn on a different part of the subject's body separately of the AR headset.

13. The method of claim 8, wherein the second sensor is disposed on the AR headset.

14. The method of claim 1, further comprising:
capturing second image data from an external image sensor separate from the AR headset; and
performing a SLAM analysis on the second image data to determine body position data of the body of the subject; and
wherein the step of determining the one or more gait attributes comprises analyzing the head-pose data and the body position data using the gait-metric prediction algorithm.

15. The method of claim 14, wherein the head-pose data and the body position data is combined prior to analyzing the head-pose data and body position data using the gait-metric prediction algorithm.

16. The method of claim 15, wherein the head-pose data and the body position data is combined using a fusion formula which applies different weights to the head-pose data and the body position data.

17. The method of claim 1, further comprising:
capturing rehab image data from the one or more image sensors of the subject performing a gait rehabilitation task for treating a gait disorder;
performing a SLAM analysis on the image data to determine rehab head-pose data of the head of the subject;
determining one or more rehab gait attributes of a gait of the subject by analyzing the rehab head-pose data using the gait-metric prediction algorithm;
analyzing the rehab gait attributes to determine feedback to the subject regarding the subject performing the gait rehabilitation task,
providing the feedback to the subject via the AR headset.

18. The method of claim 1, further comprising:
determining a gait disorder based on the gait attributes;
determining a rehabilitation task for treating the gait disorder;
providing rehabilitation task virtual content regarding the rehabilitation task to the subject via the AR headset;
capturing rehab image data from the one or more image sensors of the subject performing the gait-rehabilitation task for treating a gait disorder;
performing a SLAM analysis on the image data to determine rehab head-pose data of the head of the subject;
determining one or more rehab gait attributes of a gait of the subject by analyzing the rehab head-pose data using the gait-metric prediction algorithm;
analyzing the rehab gait attributes to determine feedback to the subject regarding the subject's performing of the rehabilitation task, providing the feedback to the subject via the AR headset.

19.-30. (canceled)

31. A system for performing a gait analysis, comprising:
an AR headset configured to be worn on a head of a subject, the AR headset comprising:
a frame structure configured to be worn on the head of the subject;
one or more image sensors carried by the frame structure; and
a display system for displaying virtual images generated by a computer system;
the computer system having at least one computer processor, memory, a storage device, and a gait-analysis software application stored on the storage device, the computer system in communication with the one or more image sensors and the display system, the computer system configured to program the system to perform a process comprising:
capturing image data from the one or more image sensors headset as the subject walks;
performing a SLAM analysis on the image data to determine head-pose data of the head of the subject; and
determining one or more gait attributes of a gait of the subject by analyzing the head-pose data using a gait-metric prediction algorithm.

32.-60. (canceled)

61. A non-transitory computer-readable medium having software instructions stored thereon, the software instructions executable by a computer processor to cause the processor to cause an AR computing system to perform a process for performing a gait analysis using an AR headset worn on the head of a subject, the process comprising:
capturing image data from one or more image sensors disposed on the AR headset as the subject walks;
performing a SLAM analysis on the image data to determine head-pose data regarding a position and location of the head of the subject; and
determining one or more gait attributes of a gait of the subject by analyzing the head-pose data using a gait-metric prediction algorithm.

62.-90. (canceled)
